(19) United States
(12) Patent Application Publication
(10) Pub. No.: US 2024/0290493 A1
(43) Pub. Date: Aug. 29, 2024

(54) SYSTEMS AND METHODS FOR EVALUATING RELIABILITY OF A PATIENT EARLY WARING SCORE

(71) Applicant: KONINKLIJKE PHILIPS N.V., EINDHOVEN (NL)

(72) Inventor: SAMAN PARVANEH, DANVERS, MA (US)

(21) Appl. No.: 18/690,106

(22) PCT Filed: Aug. 29, 2022
(86) PCT No.: PCT/EP2022/073868

§ 371 Date: Mar. 7, 2024

Related U.S. Application Data
(60) Provisional application No. 63/241,195, filed on Sep. 7, 2021.

(51) Int. Cl. G16H 50/20; G16H 10/60; G16H 50/30; G16H 50/70

(52) U.S. Cl. CPC G16H 50/20; G16H 10/60; G16H 50/30; G16H 50/70

(57) ABSTRACT

A system for evaluating the reliability of an early warning score (EWS) is provided. The system receives patient test data and determines an EWS for the patient. A real-time feature extractor extracts features from the patient test data. A reliability score evaluator generates a reliability score for the EWS by processing the extracted features through a reliability score regression model. An inference engine generates inferences based on the reliability score and the extracted features. The inferences can be displayed on a user interface. The reliability score regression model can be determined via deep learning training. The training portion of the system receives training data sets. A data annotator assigns each training data set a reliability annotation. A training feature extractor generates extracted training features from the training data sets. A deep learning trainer uses the extracted training features and the reliability annotations to generate the reliability score regression model.

### FIELD OF THE DISCLOSURE

The present disclosure is directed generally to systems and methods for evaluating the reliability of an early warning score (EWS) determined for a patient.

### BACKGROUND

Early warning scores (EWS) evaluate a risk of a patient developing a health condition before the condition occurs. EWS is determined based on patient data. The patient data may be determined through patient measurements or review of patient records. Reliability of the determined EWS depends on multiple factors, such as availability of the measurements, age of the measurements, and signal quality index (SQI) of the measurements. Due to this varying reliability, physicians receiving EWS evaluations of their patients have expressed a desire for an accompanying evaluation of the reliability of the EWS evaluation itself to aid their decision-making process regarding potential interventions and treatments. Accordingly, there is a need in the art for systems and methods to evaluate the reliability of EWS, and to provide the evaluation to a medical professional.

### SUMMARY OF THE DISCLOSURE

The present disclosure is directed generally to systems and methods for evaluating the reliability of an early warning score (EWS) determined for a patient. The systems and methods determine a reliability score for a corresponding EWS through the training and implementation of a reliability score regression model which processes features extracted from patient test data.

The system receives a variety of patient test data corresponding to a patient, and uses this patient test data to determine an EWS for the patient. A real-time feature extractor generates one or more extracted features from the patient test data. A reliability score evaluator then gencrates a reliability score for the determined EWS by processing the extracted features through a reliability score regression model. The reliability score can then be displayed on a user interface.

An inference engine can be used to generate one or more inferences based on the reliability score and the extracted features. The inferences can be displayed on a user interface, as well as provided to the medical professional by way of a notification.

The reliability score regression model can be determined via deep learning training. The training portion of the system receives a plurality of training data sets. The training data sets are provided to a data annotator to assess the reliability of the training EWS of the training data set. The data annotator assigns each training data set a reliability annotation based on the assessment. The assessment may be performed manually or automatically. A training feature extractor generates one or more extracted training features from each of the plurality of training data sets. A deep learning trainer uses the extracted training features and the reliability annotations to generate the reliability score regression model.

Generally, in one aspect, a system for evaluating an early warning score (EWS) of a patient is provided. The system includes a test data receiver. The test data receiver is configured to receive patient test data.

The system further includes an EWS evaluator. The EWS evaluator is configured to determine an EWS based on the patient test data. According to an example, at least a portion of the patient test data is collected by a patient monitor.

The system further includes a real-time feature extractor. The real-time feature extractor is configured to generate one or more extracted features from the patient test data.

The system further includes a reliability score evaluator. The reliability score evaluator is configured to generate a reliability score corresponding to the EWS based on the one or more extracted features and a reliability score regression model.

According to an example, the system further includes an inference engine. The inference engine is configured to generate one or more inferences based on the reliability score and at least one of the one or more extracted features. The inference engine may be further configured to display at least one of the one or more inferences via a user interface. The inference engine may be further configured to generate a notification corresponding to at least one of the one or more inferences.

According to a further example, the system further includes a training data receiver. The training data receiver is configured to receive a plurality of training data sets. Each of the plurality of training data sets includes a training EWS and one or more training features.

The system further includes a data annotator. The data annotator is configured to assign a reliability annotation to each of the plurality of training data sets based on the training EWS. According to an example, the data annotator assigns at least one reliability annotation based on a user input. According to another example, the data annotator assigns at least one reliability annotation based on proximity to an EWS threshold. According to a further example, the data annotator assigns at least one reliability annotation based on an EWS threshold crossing count. The EWS threshold crossing count may be determined during a predefined period. According to an even further example, the data annotator assigns at least one reliability annotation based on an EWS variability window.

The system further includes a training feature extractor. The training feature extractor is configured to generate one or more extracted training features from each of the plurality of training data sets. According to an example, the one or more extracted features include at least one of measurement availability, measurement expiration, measurement discontinuation, age of feature, feature value, short-term delta feature, long-term delta feature, and signal quality index (SQI).

According to an example, each of the one or more extracted features corresponds to one or more patient characteristics based on the patient test data. The one or more patient characteristics may include at least one of heart rate, oxygen saturation, respiratory rate, temperature, diastolic blood pressure, systolic blood pressure, patient age, pulse pressure, approximate mean arterial pressure, and shock index.

The system further includes a deep learning trainer. The deep learning trainer is configured to generate the reliability score regression model based on the reliability annotations and the one or more extracted training features.

Generally, in another aspect, a method for evaluating reliability of an EWS of a patient is provided. The method includes receiving a plurality of training data sets, wherein each of the plurality of training data sets comprises a training EWS and one or more training features. The method further includes assigning, via a data annotator, a reliability annotation to each of the plurality of training data sets. The method further includes generating, via a training feature extractor, one or more extracted training features from each of the plurality of training data sets. The method further includes generating, via a deep learning trainer, a reliability score regression model based on the reliability annotations and the one or more extracted training features. The method further includes receiving patient test data. The method further includes determining, via an EWS evaluator, an EWS based on the patient data. The method further includes gencrating, via a real-time feature extractor, one or more extracted features from the patient test data. The method further includes generating, via a reliability score evaluator, a reliability score corresponding to the EWS based on the one or more extracted features and a reliability score regression model.

In various implementations, a processor or controller may be associated with one or more storage media (generically referred to herein as “memory.” e.g., volatile and non-volatile computer memory such as RAM, PROM, EPROM, EEPROM, floppy disks, compact disks, optical disks, magnetic tape, SSD, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects as discussed herein. The terms “program” or “computer program” are used herein in a generic sense to refer to any type of computer code (e.g., software or microcode) that can be employed to program one or more processors or controllers.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a system diagram of a system for evaluating an early warning score of a patient, in accordance with an example.

FIG. 2 is a system diagram of a training subsystem for a reliability score regression model, in accordance with an example.

FIG. 3 is a user interface displaying an early warning score, a reliability score, a plurality of inferences, and a plurality of patient characteristics, in accordance with an example.

FIG. 4 is a flow chart of a method for determining a reliability score for an early warning score, in accordance with an example.

### DETAILED DESCRIPTION OF EMBODIMENTS

The present disclosure is directed generally to systems and methods for evaluating the reliability of an early warning score (EWS) determined for a patient. The systems and methods determine a reliability score for a corresponding EWS through the training and implementation of a reliability score regression model which processes features extracted from patient test data.

The system receives a variety of patient test data corresponding to a patient, and uses this patient test data to determine an EWS for the patient. The patient test data can be derived from a variety of patient measurements, as well as from patient records. The patient test data can be the basis for determining a wide array of patient characteristics, such as heart rate, oxygen saturation, respiratory rate, temperature, diastolic blood pressure, systolic blood pressure, patient age, pulse pressure, approximate mean arterial pressure, and shock index.

A real-time feature extractor generates one or more extracted features from the patient test data. The extracted features describe the patient test data and patient measurements used to determine the patient characteristics: accordingly, the extracted features are key indicators of the reliability of the determined EWS. The extracted features can be a wide array of properties, such as measurement availability, measurement expiration, measurement discontinuation, age of feature, feature value, short-term delta feature, long-term delta feature, and signal quality index (SQI).

A reliability score evaluator then generates a reliability score for the determined EWS by processing the extracted features through a reliability score regression model. The reliability score can be normalized such that it is a number between 0 and 1, where 0 corresponds to the most unreliable EWS, and 1 corresponds to the most reliable EWS. The reliability score can then be displayed on a user interface, or provided to a medical professional via any other practical means.

An inference engine can be used to generate one or more inferences based on the reliability score and the extracted features. For example, if the reliability score is low, and the extracted features indicate a low SQI for a heart rate measurement, an inference could suggest that the medical professional check electrocardiogram leads and/or electrodes, and then re-take the measurement. The inferences can be displayed on a user interface, as well as provided to the medical professional by way of a notification. The notification may be visual and/or audible.

The reliability score regression model can be determined via deep learning training. The training portion of the system receives a plurality of training data sets. Each training data set includes a training EWS and one or more training features.

The training data sets are provided to a data annotator to assess the reliability of the training EWS of the training data set. The data annotator assigns each training data set a reliability annotation based on the assessment. The assessment may be performed manually, such as by a medical professional reviewing the training EWS, and inputting their assessment of the reliability of the training EWS. In other examples, the assessment is performed automatically, such as by evaluating the proximity of the training EWS to an EWS threshold, by counting the times a series of training EWS crosses the EWS threshold, or by evaluating the variability of a series of training EWS based on an EWS variability window.

A training feature extractor generates one or more extracted training features from each of the plurality of training data sets. A deep learning trainer uses the extracted training features and the reliability annotations to generate the reliability score regression model.

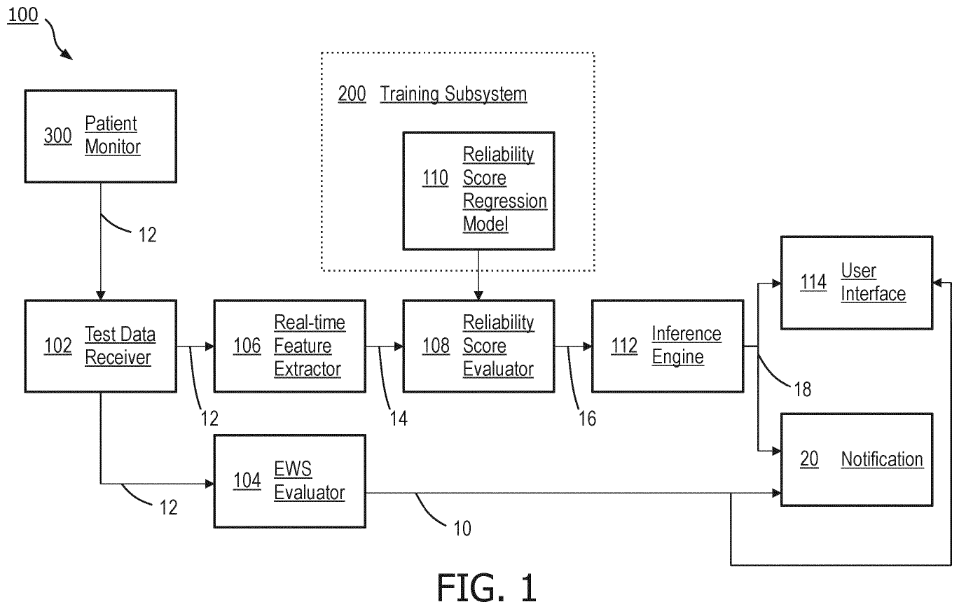

FIG. 1 shows a system diagram of a system 100 for evaluating an EWS 10 of a patient. The system 100 includes a test data receiver 102. The test data receiver 102 receives patient test data 12 from one or more sources. The patient test data 12 can be derived from a variety of patient measurements and/or patient records. For example, the patient test data 12 can include patient measurements such as heart rate, blood pressure, electrocardiogram signals, blood oxygen level, etc. These measurements can be taken by a wide array of equipment communicatively coupled to a patient monitor 300. The patient monitor 300 can then pass on these measurements, in the form of patient test data 12, to the test data receiver 102. The test data receiver 102 can also receive a variety of patient records, which can include demographic information (age, ethnicity, etc.) and historical information regarding the health history of the patient and their family. The test data receiver 102 can be configured as a wired and/or wireless communication component or sub-component.

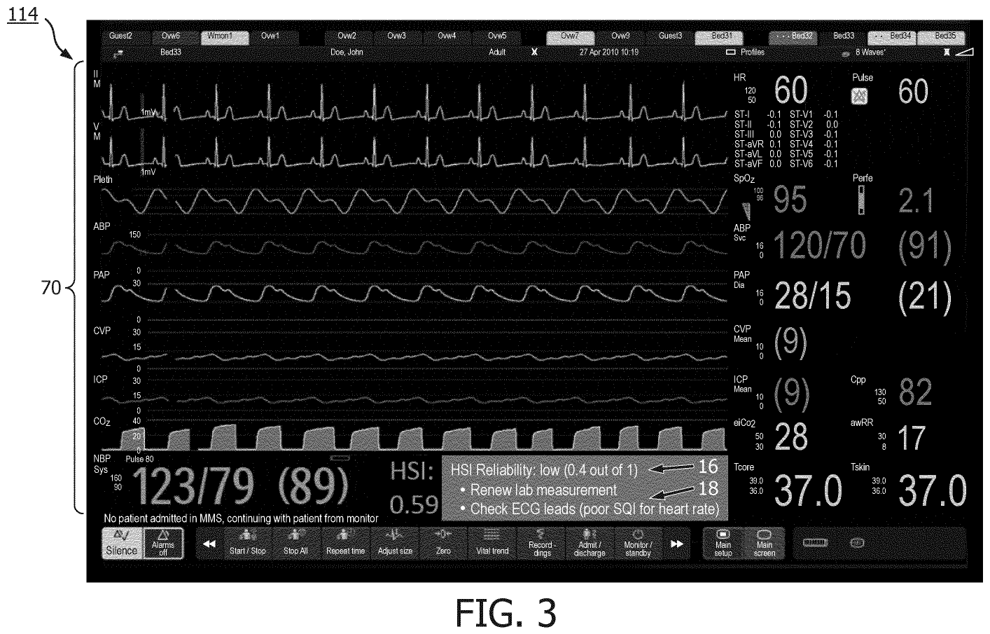

The test data receiver 102 then conveys the patient test data 12 to an EWS evaluator 104. The EWS evaluator 104 determines an EWS 10 based on the patient test data 12. EWS 10 represents the risk of developing a health condition before occurrence. For example, and as shown in FIG. 3, the EWS 10 can be a hemodynamic stability index representing the risk of the patient developing hemodynamic instability. The EWS 10 can be normalized to be on a scale of 0 to 1, with 0 representing the lowest risk, and 1 representing the highest risk. While EWS 10 is an important prediction and treatment tool, the reliability of the EWS 10 can fluctuate based on a variety of factors. Accordingly, by providing a reliability score 16 associated with the EWS 10, a medical professional can quickly assess the predictive risks facing the patient, as well as the reliability of that assessment, before taking action regarding treatments and/or other interventions. As shown in FIG. 3, the EWS 10 can be displayed on a user interface 18. In FIG. 3, the EWS 10 is a hemodynamic stability index score of 0.59.

The EWS 10 can also be provided to a medical professional via a notification 20, such as a visual and/or audio notification. The display and/or notification 20 of the EWS 10 may correspond to the severity of the EWS 10.

The test data receiver 102 also conveys the patient test data 12 to a real-time feature extractor 106. The real-time feature extractor 106 “extracts” features regarding the patient test data 12 which relate to the reliability of the EWS 10. The extracted features can be a wide array of properties, such as measurement availability, measurement expiration, measurement discontinuation, age of feature, feature value, short-term delta feature, long-term delta feature, and SQI. Some or all of these extracted features can correspond to one or more patient characteristics 70, such as heart rate, oxygen saturation, respiratory rate, temperature, diastolic blood pressure, systolic blood pressure, patient age, pulse pressure, approximate mean arterial pressure, and shock index.

Measurement availability can be a binary indicator of whether a measurement (such as heart rate) is currently available. Similarly, measurement expiration can be a binary indicator of whether a measurement is currently available. Further, measurement discontinuation can be a binary indicator of whether a measurement has stopped collecting data. Age of feature indicates the time duration from the last measurement corresponding to the feature (such as 60 seconds from the last oxygen saturation measurement). Feature value indicates the value of the measurement corresponding to the feature (such as a heart rate of 80 beats per minute). Short-term delta and long-term delta indicate the variability of a measurement (such as temperature) over a short or long period. SQI indicates the signal quality associated with a measurement corresponding to the feature on a scale of 0 to 1. By analyzing these extracted features 14 in the aggregate using a reliability score evaluator 108, the system 100 can quickly determine a reliability score 16 for the EWS 10.

The real-time feature extractor 106 conveys the extracted features 14 to a reliability score evaluator 108. The reliability score evaluator 108 calculates a reliability score 16 by processing the extracted features 14 through a reliability score regression model 110. As will be explained in greater detail below, the reliability score regression model 110 is trained by training subsystem 200 using a deep learning trainer 206 and training data sets 50. Accordingly, the reliability score regression model 110 learns how certain extracted features 16 (and their associated values) correlate to the reliability of the EWS 10. For example, a high degree of short-term variability (delta) of a diastolic blood pressure measurement may correspond to lower reliability of an EWS 10 related to hemodynamic stability. Thus, this measurement would result in a lower reliability score 16. Conversely, a low degree of long-term variability of the diastolic blood pressure measurement may correspond to higher reliability of the EWS 10 related to hemodynamic stability, resulting in a higher reliability score. On the other hand, the value of the temperature of the patient may not be particularly indicative of the reliability of the EWS 10 related to hemodynamic stability, and thus does not result in an increased or decrease reliability score 16.

The reliability score 16 may be normalized such that the reliability score 16 is between 0 and 1, where a reliability score 16 of 0 represents the lowest degree of reliability, while a reliability score 16 of 1 represents the highest. For example, FIG. 3 shows a user interface 114 displaying a hemodynamic score index with a reliability score 16 of 0.4 out of 1. Thus, the hemodynamic score index has a slightly-below medium reliability.

Once the reliability score 16 is calculated, an inference engine 112 then generates one or more inferences 18 based on the reliability score 16 and the extracted features 14. An inference 18 can provide more detail to a medical professional regarding the reliability score 16, as well as provide the medical professional with suggestions for improving the reliability score 16. For example, FIG. 3 shows a user interface 114 displaying two inferences 18, “Renew lab measurement” and “Check ECG leads (poor SQI for heart rate).” These inferences 18 offer suggestions for the medical professional to improve the hemodynamic score index from 0.4 to a number indicating higher reliability by checking the ECGs leads and renewing the measurements.

As shown in FIG. 3, the inferences 18 can be displayed on a user interface 114. The user interface 114 can include a wide array of other information, such as the reliability score 16 and one or more patient characteristics 70. In one example, the user interface 114 is a touch screen embedded in a patient monitor 300. Alternatively, the user interface 114 can be a component of a smartphone, personal computer, tablet computer, or any other appropriate device.

Further, the inference 18 can be provided to the medical professional as one or more notifications 20, such as a visual and/or audio notification. The visual/or audio notifications 20 of the inferences 18 may correspond to the severity of the inference 18. For example, the “Check ECG leads (poor SQI for heart rate)” inference 18 may correspond to a notification 20 of an audible tone (or series of audible tones) emitted by the patient monitor 300. In this example, the notification 20 can also include a blinking LED or set of pixels in the user interface 114. The notification 20 can also be conveyed to a device operated by the medical professional, such as a smartphone, personal computer, or tablet computer.

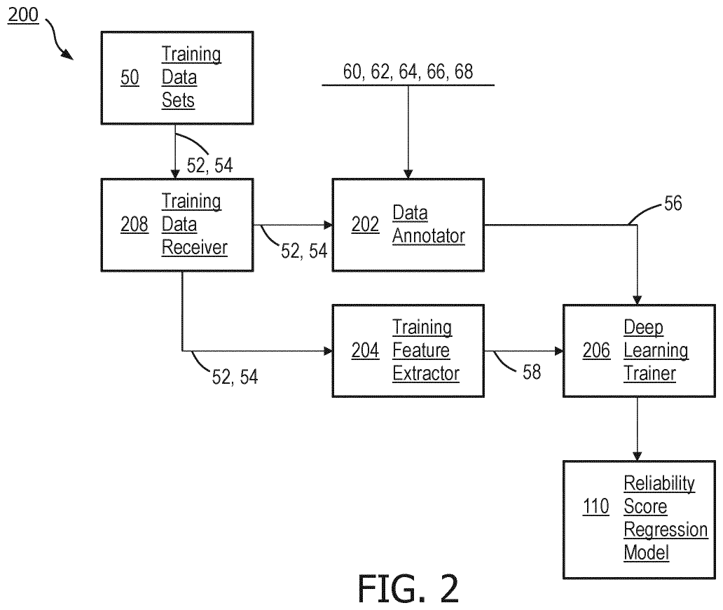

FIG. 2 shows a system diagram of the training subsystem 200 for the reliability score regression model 110 via deep learning training. As shown in FIG. 2, a training data receiver 208 receives a plurality of training data sets 50. Each training data set 50 includes a training EWS 52 and one or more training features 54. As with the extracted features 14, the training features 54 can include measurement availability, measurement expiration, measurement discontinuation, age of feature, feature value, short-term delta feature, long-term delta feature, and SQI.

As shown in FIG. 2, the training data sets 50 are provided to a data annotator 202. The data annotator 202 is configured to assess the reliability of the training EWS 52 of the training data sct 50. The data annotator 202 assigns each training data set 50 a reliability annotation 56 based on the assessment. The reliability annotation 56 may be binary (reliable or not), or it may be in the form of a numeric scale. The assessment may be performed manually, such as by an expert medical professional reviewing the training EWS 52, and inputting their assessment of the reliability of the training EWS 52. For example, an expert medical professional may determine the training EWS 52 to be inaccurate based on the variability of the training EWS 52 over a period of time. Further, and inaccuracy assessment may be based on an apparent mismatch between the training EWS 52 and training features 54 (such as feature values).

Manual assessment can be time consuming and expensive. Accordingly, in other examples, the data annotator 202 performs assessments automatically according to one or more programmable criteria. In one example, the data annotator 202 assesses the reliability of a training EWS 52 based on the proximity of the training EWS 52 to an EWS threshold 60. In this example, reliability annotation 56 can correspond to a decrease in reliability if the training EWS 52 is close to, or exceeds, the EWS threshold 60. The EWS threshold 60 may be an “optimum cut-off” for the training EWS 52.

In another example, the data annotator 202 assesses the reliability of a series of training EWS 52 by counting the instances of the training EWS 52 crossing the EWS threshold 60 during a predetermined period 66, such as 30 minutes. In a further example, the data annotator 202 assesses the reliability of a series of training EWS 52 by evaluating the variability of EWS training scores 52 during an EWS variability window 68, such as 30 minutes.

Any aspect of the aforementioned automatic data annotator 202 may be adjusted hospital and/or clinicians preferences or definition of reliability.

While the data annotator 202 is assigning reliability annotations 56, a training feature extractor 204 generates one or more extracted training features 58 from each of the plurality of training data sets 50. The training feature extractor 204 operates similar to the real-time feature extractor 106 shown in FIG. 1.

The reliability annotations 56 and the extracted training features 58 are provided to a deep learning trainer 206. The deep learning trainer 206 evaluates the correlations between the extracted training features 58 and the reliability annotation 56 of each set. For example, the unavailability of an important measurement, or a low SQI for the important measurement, may be highly correlative to low reliability annotations 56. Conversely, the age of the patient may not be correlative to the value of the corresponding relatability annotations 56. The deep learning trainer 206 uses these correlations to generate a reliability score regression model 110. As described above, the reliability score regression model 110 may then be used to generate a reliability score 16 for an EWS 10 based on one or more extracted features 14. For example, in accordance with the earlier example, if one of the extracted features 14 indicates an important measurement (such as heart rate) is unavailable, or the measurement has low SQI value, the reliability score regression model 110 may generate a lower reliability score 16. In one example, the reliability score regression model 110 is an XGBoost regressor allowing for an estimation of a reliability score 16 based on one or more top contributing extracted features 14.

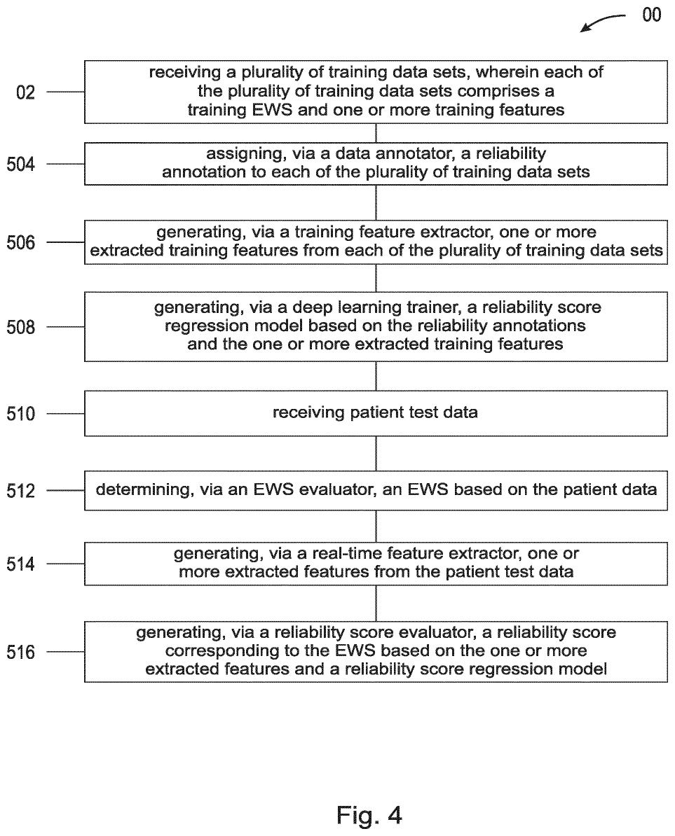

Generally, in another aspect, and with reference to FIG. 4, a method 500 for evaluating reliability of an EWS of a patient is provided. The method 500 includes receiving 502 a plurality of training data sets, wherein each of the plurality of training data sets comprises a training EWS and one or more training features. The method 500 further includes assigning 504, via a data annotator, a reliability annotation to each of the plurality of training data sets. The method 500 further includes generating 506, via a training feature extractor, one or more extracted training features from each of the plurality of training data sets. The method 500 further includes generating 508, via a deep learning trainer, a reliability score regression model based on the reliability annotations and the one or more extracted training features. The method 500 further includes receiving 510 patient test data. The method 500 further includes determining 512, via an EWS evaluator, an EWS based on the patient data. The method 500 further includes generating 514, via a real-time feature extractor, one or more extracted features from the patient test data. The method 500 further includes generating 516, via a reliability score evaluator, a reliability score corresponding to the EWS based on the one or more extracted features and a reliability score regression model.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of.” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “cither,” “one of.” “only one of.” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The above-described examples of the described subject matter can be implemented in any of numerous ways. For example, some aspects may be implemented using hardware, software or a combination thereof. When any aspect is implemented at least in part in software, the software code can be executed on any suitable processor or collection of processors, whether provided in a single device or computer or distributed among multiple devices/computers.

The present disclosure may be implemented as a system, a method, and/or a computer program product at any possible technical detail level of integration. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, configuration data for integrated circuitry, or cither source code or object code written in any combination of one or more programming languages, including an object oriented programming language such as Smalltalk. C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some examples, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to examples of the disclosure. It will be understood that each block of the flowchart illustrations and/or block diagrams, and combinations of blocks in the flowchart illustrations and/or block diagrams, can be implemented by computer readable program instructions.

The computer readable program instructions may be provided to a processor of a, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various examples of the present disclosure. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Other implementations are within the scope of the following claims and other claims to which the applicant may be entitled.

While various examples have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the examples described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings is/are used. Those skilled in the art will recognize, or be able to ascertain using no more than routine experimentation, many equivalents to the specific examples described herein. It is, therefore, to be understood that the foregoing examples are presented by way of example only and that, within the scope of the appended claims and equivalents thereto, examples may be practiced otherwise than as specifically described and claimed. Examples of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

1. A system for evaluating an early warning score (EWS) of a patient, comprising:
a test data receiver configured to receive patient test data;
an EWS evaluator configured to determine an EWS based on the patient test data;
a real-time feature extractor configured to generate one or more extracted features from the patient test data; and
a reliability score evaluator configured to generate a reliability score corresponding to the EWS based on the one or more extracted features and a reliability score regression model.

2. The system of claim 1, further comprising an inference engine configured to generate one or more inferences based on the reliability score and at least one of the one or more extracted features.

3. The system of claim 2, wherein the inference engine is further configured to display at least one of the one or more inferences via a user interface.

4. The system of claim 2, wherein the inference engine is further configured to generate a notification corresponding to at least one of the one or more inferences.

5. The system of claim 1, further comprising:
a training data receiver configured to receive a plurality of training data sets, wherein each of the plurality of training data sets comprises a training EWS and one or more training features;
a data annotator configured to assign a reliability annotation to each of the plurality of training data sets based on the training EWS;
a training feature extractor configured to generate one or more extracted training features from each of the plurality of training data sets; and
a deep learning trainer configured to generate the reliability score regression model based on the reliability annotations and the one or more extracted training features.

6. The system of claim 5, wherein the data annotator assigns at least one reliability annotation based on a user input.

7. The system of claim 5, wherein the data annotator assigns at least one reliability annotation based on proximity to an EWS threshold.

8. The system of claim 5, wherein the data annotator assigns at least one reliability annotation based on an EWS threshold crossing count.

9. The system of claim 8, wherein the EWS threshold crossing count is determined during a predefined period.

10. The system of claim 5, wherein the data annotator assigns at least one reliability annotation based on an EWS variability window.

11. The system of claim 1, wherein each of the one or more extracted features corresponds to one or more patient characteristics based on the patient test data.

12. The system of claim 11, wherein the one or more patient characteristics comprise at least one of heart rate, oxygen saturation, respiratory rate, temperature, diastolic blood pressure, systolic blood pressure, patient age, pulse pressure, approximate mean arterial pressure, and shock index.

13. The system of claim 1, wherein the one or more extracted features comprise at least one of measurement availability, measurement expiration, measurement discontinuation, age of feature, feature value, short-term delta feature, long-term delta feature, and signal quality index (SQI).

14. The system of claim 1, wherein at least a portion of the patient test data is collected by a patient monitor.

15. A method for evaluating reliability of an early warning score (EWS) of a patient, comprising:
receiving a plurality of training data sets, wherein each of the plurality of training data sets comprises a training EWS and one or more training features;
assigning, via a data annotator, a reliability annotation to each of the plurality of training data sets;
generating, via a training feature extractor, one or more extracted training features from each of the plurality of training data sets;
generating, via a deep learning trainer, a reliability score regression model based on the reliability annotations and the one or more extracted training features;
receiving patient test data;
determining, via an EWS evaluator, an EWS based on the patient data;
generating, via a real-time feature extractor, one or more extracted features from the patient test data; and
generating, via a reliability score evaluator, a reliability score corresponding to the EWS based on the one or more extracted features and a reliability score regression model.
